(19) United States
(12) Patent Application Publication
(10) Pub. No.: US 2012/0178146 A1
(43) Pub. Date: Jul. 12, 2012

(54) BACTERIA CAPABLE OF DEGRADING MULTIPLE PETROLEUM COMPOUNDS IN SOLUTION IN AQUEOUS EFFLUENTS AND PROCESS FOR TREATING SAID EFFLUENTS

(75) Inventors: Marc Auffret, Pontivy (FR); Francoise Fayolle-Guichard, Paris (FR); Gerald Thouand, La Roche Sur Yon (FR); Charles W. Greer, Pointe-Claire (CA); Diane Labbe, Pierrefonds (CA)

(73) Assignee: IFP Energies Nouvelles, Rueil-Malmaison Cedex (FR)

(21) Appl. No.: 13/262,647

(22) PCT Filed: Mar. 26, 2010
(86) PCT No.: PCT/FR2010/000259

§ 371 Date: Mar. 27, 2012

(30) Foreign Application Priority Data
Apr. 3, 2009 (FR) 09/01.637

(51) Int. Cl. C12N 1/20; C12S 3/00

(52) U.S. Cl. 435/252.1; 435/267

(57) ABSTRACT

This invention relates to new Rhodococcus wratislaviensis CNCM I-4088 bacteria or Rhodococcus aetherivorans CNCM I-4089 bacteria that can degrade multiple petroleum compounds in solution in aqueous effluents.

The invention also relates to a process for treating aqueous effluents comprising a complex mixture of substances containing native hydrocarbons of gasolines and additives that are present in gasolines or diesel fuel, in which process said bacteria are grown under aerobic conditions in the presence of a growth substrate containing said mixture as a carbon source, and said mixture is at least partially degraded by the bacteria down to the final degradation products—carbon dioxide, water and biomass.

### FIELD OF THE INVENTION

This invention relates to microorganisms that can degrade complex mixtures of hydrocarbons in solution in water.

It applies, in particular, to the water treatment industry for the most part, but also to the treatment of soils and wastes polluted by these compounds.

### EXAMINATION OF PRIOR ART

It is known that gasolines and diesel fuels are complex mixtures of different chemical compounds. Moreover, certain compounds are added to gasolines and diesel fuel after the refining process in order to respond to motorists' particular specifications. This is the case especially with oxygenated additives or ether fuels: methyl-tert-butyl ether (hereafter referred to by the term MTBE) is one of the ethers that can be used as an oxygenated additive in unleaded gasolines for the purpose of increasing their octane number as well as ethyl-tert-butyl ether (hereafter referred to as ETBE), which has been used preferentially for several years in France and also in other European countries because of its qualification as a biofuel. These compounds can be added to gasolines at a rate of 15% (v/v). Other molecules are often added to diesel fuel. For example, 2-ethyl hexyl nitrate (hereafter referred to by the term 2-EHN), which can be added to diesel fuel at a rate of 0.5% (v/v) to meet the specifications regarding the cetane number, will be cited.

The transport of hydrocarbons, by overland or sea routes, presents numerous risks of accidents. Overland transport via pipelines, which is generally considered safer than by truck, train, or tanker, can nevertheless result in cases of pollution. It has been estimated that the quantity of hydrocarbons spilled during transport via underground pipelines is approximately 60 m³/1000 km of pipe (Académie des Sciences, 2000 [Academy of Sciences, 2000]). Moreover, incidents of ground pollution by hydrocarbons are due to truck or train accidents during transport, accidents while filling service station tanks, and leaks in service station storage tanks or at industrial sites. In addition to these major sources of pollution by hydrocarbons, chronic pollution occurs when vehicle gas tanks are filled in service stations or because of leaks in vehicle gas tanks. In these last two cases, this discharge to ground waters is small in quantity, but chronic, and also has a significant impact.

Among the gasoline compounds, all do not have the same toxicity and/or biodegradability, and this will determine their future in the environment. Benzene, for example, which is one of the monoaromatic gasoline compounds, is a compound that is very toxic, but easily degraded in aerobiosis. Among the native gasoline compounds that are recalcitrant to biodegradation, 2,2,4-trimethylpentane (hereafter referred to by the term isooctane) or cyclohexane, the toxicity levels of which are less than those of benzene, can be cited.

Moreover, the increasing use of additives such as MTBE, ETBE, or 2-EHN results in large stored and transported volumes, by themselves or in a mixture in gasolines or diesel fuel. The poor biodegradability of these additives is an established fact. It is therefore necessary to know the future of these compounds in the event of accidental spilling of the product itself or of gasolines or diesel fuel with additives, because these discharges into the environment lead to pollution of soils and subterranean or surface waters.

Literature regarding biodegradation of gasoline or alkane compounds by microorganisms is substantial (Microbiologie pétrolière [Petroleum Microbiology] by JP Vandecasteele, 2005 Editions Technip). By contrast, fewer studies are devoted to additives (MTBE, ETBE, 2-EHN), mainly due to the recalcitrance of these molecules to biodegradation, or to the study of biodegradation of complex mixtures that end up dissolving in water in the event of hydrocarbon spills and due to the difficulty of analyzing complex mixtures. In these cases, the implementation of microcosms, the composition of which is not generally known and undoubtedly not determined by biopurification processes (biofilters, etc.), is often reported in literature.

Few publications have carried out broad studies of the degradation capacities of a given bacterial strain with respect to a wide range of hydrocarbons or additives, which are added to them either by themselves or in a mixture. It is possible to cite, for example, the publication Solano-Serena et al., 2000, Applied and Environmental Microbiology, 66: 2392-2399, which describes the capacities of the bacterial strain Mycobacterium austroafricanum, among others, to degrade isooctane. Data on the capacities of isolated microorganisms to degrade a wide range of hydrocarbons are generally not available.

It therefore appears necessary to find and identify new microorganisms that can biodegrade complex mixtures of substances containing native hydrocarbons and additives (such as, for example, MTBE, ETBE, and 2-EHN), which can reach aquifer layers in cases of pollution, and to study their use in water treatment processes, thereby allowing significant decreases in residual concentrations of the above-described pollutants in urban or industrial waste water or in contaminated aquifer layers, referred to by the general name effluents, contaminated by these compounds.

This invention falls within this framework.

### SUMMARY PRESENTATION OF THE INVENTION

This invention relates to two bacterial strains isolated from a bacterial microcosm and demonstrating significant capacities for biodegradation of a complex mixture of hydrocarbons in solution in water.

Also described is a process for treating aqueous effluents containing at least a complex mixture of hydrocarbons in which the two bacterial strains are grown.

### DETAILED DESCRIPTION OF THE INVENTION

This invention relates to a process for treating aqueous effluents comprising a complex mixture of substances containing native hydrocarbons of gasolines and additives that are present in gasolines or diesel fuel in which at least one bacterium, selected from among the Rhodococcus wratislaviensis CNCM I-4088 and Rhodococcus aetherivorans CNCM I-4089 bacteria, is grown under aerobic conditions in the presence of a growth substrate that contains said mixture as a carbon source, and said mixture is at least partially degraded by the bacteria down to the final degradation products—carbon dioxide, water and biomass.

The invention also relates to the new Rhodococcus wratislaviensis I-4088 and Rhodococcus aetherivorans I-4089 bacteria, deposited at the Institut Pasteur [Pasteur Institute] on Nov. 20, 2008. (CNCM [la Collection Nationale de Cultures de Microorganismes/National Collection of Microorganism Cultures] of the Institut Pasteur, 25, rue du Docteur Roux, F-75724 PARIS CEDEX 15).

The complex mixture of substances containing native hydrocarbons of gasolines and additives present in the gasolines or diesel fuel is a mixture of 16 different compounds with equal mass concentrations. It comprises, in particular, compounds that are selected from among alkanes, monoaromatic hydrocarbons, polycyclic aromatic hydrocarbons, ethers or nitrates.

Preferably, the mixture comprises the following 16 compounds: octane, hexadecane, benzene, ethylbenzene, toluene, m-xylene, p-xylene, o-xylene, cyclohexanol, tert-butanol (hereafter referred to by the term TBA), cyclohexane, isooctane, MTBE, ETBE, 2-ethyl hexyl nitrate (hereafter referred to by the term 2-EHN), and naphthalene.

According to a preferred embodiment of the treatment process according to the invention, the two bacteria Rhodococcus wratislaviensis CNCM I-4088 or Rhodococcus aetherivorans CNCM I-4089 are grown under aerobic conditions in the presence of a growth substrate containing said mixture as a carbon source, and said mixture is at least partially degraded by the bacteria down to the final degradation products—carbon dioxide, water, and biomass.

The two bacteria Rhodococcus wratislaviensis CNCM I-4088 and Rhodococcus aetherivorans CNCM I-4089 have been tested by themselves or in co-culture for their capacities for degradation of the mixture of the 16 compounds described above.

When the mixture of compounds above is provided to Rhodococcus wratislaviensis CNCM I-4088, the bacterium proves capable of degrading—completely—11 of the 16 compounds present. Three other compounds, MTBE, 2-EHN, and ETBE, are significantly degraded (degradation capacity of greater than 50%). The isooctane is degraded only slightly (degradation capacity of 26.3%). The TBA is not degraded by this bacterium. Moreover, during the degradation of ETBE and MTBE, the bacterium produces TBA, which is added to that provided in the mixture, since it is not degraded by this bacterium and accumulates in the growth medium. This fact is well known to one skilled in the art.

The degradation capacities of Rhodococcus aetherivorans CNCM I-4089 are more limited, but nevertheless remain advantageous. This bacterium totally degrades two of the compounds: hexadecane and, in particular, ETBE. It partially degrades, with a degradation capacity of less that 50%, ethylbenzene, MTBE, and 2-EHN. The other compounds are not degraded. As in the case of Rhodococcus wratislaviensis CNCM I-4088, the bacterium produces TBA during degradation of ETBE and MTBE, which becomes added to that provided in the mixture.

These two bacteria can be advantageously combined to constitute a co-culture, the initial composition of which is determined by the operator, thereby facilitating his control and tracking. In this case, 13 out of 16 compounds in the mixture are totally degraded, 2-EHN is significantly degraded (degradation capacity of greater than 50%), and isooctane to a lesser degree (degradation capacity of less than 50%). In this case, TBA accumulates in the medium as described in the case of the single strains.

Because of the lack of TBA degradation capacities in the two bacteria, it is very advantageous to supplement the bacterial consortium by adding to it a third bacterium previously described by the applicant because of its capacity to grow on TBA. This bacterium, previously called Pseudomonas cepacia CNCM I-2052 and which was the subject of a previous patent EP-B-1 099 753, was recently renamed Aquincola tertiaricarbonis CNCM I-2052 following a change in the classification of microorganisms.

Very preferably, in the treatment process according to the invention, a consortium containing the three bacteria Rhodococcus wratislaviensis CNCM I-4088, Rhodococcus aetherivorans CNCM I-4089, and Aquincola tertiaricarbonis CNCM I-2052 is grown under aerobic conditions in the presence of a growth substrate containing said mixture as a carbon source, and said mixture is at least partially degraded by the bacteria down to the final degradation products—carbon dioxide, water, and biomass.

In cases of co-cultures, the initial composition of the bacteria mixture is such that an equal quantity of each bacterium is placed in the medium.

Thus, according to this latter embodiment, the mixture of 16 compounds is degraded in its entirety, with the exception of isooctane, which is only partially degraded. The use of such a consortium for removing pollution from contaminated effluents by various types of hydrocarbons is advantageous because each of the elements of the consortium is well identified, which allows the stability of the consortium during the process for removing pollution to be ensured and also allows its evolution to be monitored.

Other objects of this invention are the new bacteria Rhodococcus wratislaviensis CNCM I-4088 and Rhodococcus aetherivorans CNCM I-4089.

These bacteria were isolated from a microcosm originating from different environments, which was transplanted successively three times on a minimal medium containing the mixture of 16 compounds described above as a carbon source. This protocol was carried out according to microorganism enrichment techniques that are well known to one skilled in the art.

After these specific enrichment stages, the two resultant bacterial strains were isolated in Petri dishes containing rich media conventionally used by one skilled in the art (Trypticase/soy medium or also called TS in abbreviated form). These bacteria were then identified according to their 16S rRNA gene sequence and by comparison with bacterial DNA data banks; then, they were tested for their capacities to degrade the 16 compounds of the mixture.

It is necessary to note that the so-called “two-phase” system, in which the pollutants are brought to the dissolved state in a third solvent, such as, for example, silicone or 2,2,4,4,6,8,8-heptamethyl-nonane, also called HMN, can be very advantageous for determining the degradation capacities of these bacteria.

The use of these bacteria for the continuous treatment of effluents polluted by hydrocarbons and their additives can be achieved by, for example, developing the bacterium or bacterial consortium on a mineral or organic substrate in a biofilter or biobarrier system of adequate volume, by introducing effluents to be treated in the presence of air or oxygen into the biofilter or biobarrier, and by drawing off the effluent with a reduced concentration of chemical substances.

The bacterium or bacterial consortium can be added as inoculum in any other system adapted to water or soil (biobarrier) treatment, and in particular to waste water purification plant sludge.

The scope of the invention will be better understood by reading the various examples presented in detail below.

### EXAMPLES

### Example 1

### Degradation of a Mixture of Hydrocarbons and Gasoline or Diesel Fuel Additives by a Bacterial Microcosm that Comes from the Environment

A microcosm is created by mixing different samples coming from the environment in order to obtain maximum degradation capacities. The microcosm was created by mixing samples having 4 different origins (Table 1).

TABLE 1
Origin of Samples from the Environment

| Samples | Origin |
|---|---|
| Waste water purification plant | Domestic waste water treatment plant |
| sludge | (France) |
| Deep soil highly polluted by | Service station (France) |
| hydrocarbons | |
| Surface soil slightly polluted by | Service station (France) |
| hydrocarbons | |
| Unpolluted soil | Forest (France) |

Each sample is filtered with a 0.22 μm filter so as to retain, as much as possible, only the microorganisms and to eliminate additional substrates that would skew the results of the biodegradation test. The microcosm that results from the mixture of these 4 filtered samples constitutes the bacterial inoculum that was cultivated in the MM medium (150 ml) in a 500 ml Schott flask.

The composition of the MM medium is as follows:

| | | |
|---|---|---|
| KH₂PO₄ | 1.4 | g |
| K₂HPO₄ | 1.7 | g |
| NH₄NO₃ | 1.5 | g |
| MgSO₄, 7 H₂O | 0.5 | g |
| CaCl₂, 2 H₂O | 0.04 | g |
| FeSO₄, 7 H₂O | 0.001 | g |
| Concentrated vitamin solution | 1 | mL |
| Concentrated oligo-element solution | 1 | mL |

| | |
|---|---|
| H₂O | q.s.p. [quantity sufficient |
| | for] 1 liter |

The concentrated vitamin solution has the following composition for 1 liter of distilled water:

| | | |
|---|---|---|
| Biotin | 200 | mg |
| Riboflavin | 50 | mg |
| Nicotinamic acid | 50 | mg |
| Pantothenate | 50 | mg |
| p-Aminobenzoic acid | 50 | mg |
| Folic acid | 20 | mg |
| Thiamine | 15 | mg |
| Cyanocobalamin | 1.5 | mg |

The concentrated solution of oligo-elements has the following composition for 1 liter of distilled water:

| | | |
|---|---|---|
| CuSO₄, 5 H₂O | 0.1 | g |
| MnSO₄, 2 H₂O | 1 | g |
| ZnSO₄, 7 H₂O | 1 | g |
| AlCl₃, 6 H₂O | 0.4 | g |
| NiCl₂, 6 H₂O | 0.25 | g |
| H₃BO₃ | 0.1 | g |
| CoCl₂, 6 H₂O | 1 | g |
| Na₂MoO₄, 2 H₂O | 1 | g |
| Na₂WO₄, 2 H₂O₂ | 1 | g |

The final pH of the medium is 6.8.

The carbon source provided is made up of a mixture of hydrocarbons and additives of gasolines or diesel fuel at a rate of 23 μl of the mother solution of the mixture described in Table 2. In this table, the final concentrations of these compounds that were obtained upon contact with water of a “type 7000” gasoline at the pump outlet are generally much lower than the concentrations used in the biodegradation test. The only cases where the concentration is higher are those of alcohols (TBA and cyclohexanol) and ethers (MTBE and ETBE), since these compounds are very soluble in water.

TABLE 2
Composition of the Mixture of 16 Compounds that Make Up the Carbon Source.

| | | Quantity | Final Concentration after | | Concentration Obtained | |
|---|---|---|---|---|---|---|
| | Volume Added | Introduced | Addition of 23 μl of | | after Contact with | |
| Chemical | to the Mother | into the Mother | Mother Solution into the | | Water of Fuel Fractions | |
| Compound | Solution (ml) | Solution (g) | Culture Flask (mg · L⁻¹) | | with Additives | |
| Octane | 1.9 | 1.285 | 7.9 | mg · L⁻¹ | 1.19 | μg · L⁻¹ |
| Hexadecane | 1.74 | 1.1886 | 7.4 | mg · L⁻¹ | # 0 | |
| Isooctane | 1.94 | 1.275 | 7.9 | mg · L⁻¹ | 108 | μg · L⁻¹ |
| Benzene | 1.52 | 1.1666 | 7.2 | mg · L⁻¹ | 697 | μg · L⁻¹ |
| Toluene | 1.54 | 1.167 | 7.2 | mg · L⁻¹ | 66 | mg · L⁻¹ |
| Ethylbenzene | 1.54 | 1.1372 | 7.1 | mg · L⁻¹ | 1741 | μg · L⁻¹ |
| o-Xylene | 1.52 | 1.1392 | 7.1 | mg · L⁻¹ | 3454 | μg · L⁻¹ |
| m-Xylene | 1.56 | 1.1285 | 7 | mg · L⁻¹ | 7467 | μg · L⁻¹ |
| p-Xylene | 1.56 | 1.1503 | 7.1 | mg · L⁻¹ | 2200 | μg · L⁻¹ |
| Naphthalene | NA | 1.34 | 8.3 | mg · L⁻¹ | 197 | μg · L⁻¹ |
| MTBE * | 1.81 | 1.2693 | 7.9 | mg · L⁻¹ | 3.8 | g · L⁻¹ |
| ETBE ** | 1.81 | 1.2955 | 8 | mg · L⁻¹ | 1.38 | g · L⁻¹ |
| TBA | 1.7 | 1.17 | 7.3 | mg · L⁻¹ | — | |
| Cyclohexane | 1.72 | 1.2441 | 7.7 | mg · L⁻¹ | 134 | μg · L⁻¹ |
| Cyclohexanol | 1.4 | 1.0929 | 6.8 | mg · L⁻¹ | — | |
| 2-EHN *** | 1.48 | 1.32 | 8.2 | mg · L⁻¹ | 41 | μg · L⁻¹ |

* Solubility of MTBE when added to gasoline at a rate of 7%
** Solubility of ETBE when added to gasoline at a rate of 12%
*** Solubility of 2-EHN when added to a diesel fuel at a rate of 0.5%

Several identical cultures are incubated while being stirred at 30° C. These cultures constitute the Mix1. Metering of residual substrates is carried out at regular intervals by extraction with pentane containing 1,1,2-TCA (or 1,1,2-trichloroethane) as an internal standard for the entirety of a flask. The pentane, after extraction of the residual substrates, is injected by gas phase chromatography with a flame ionization detector GPC/FID on a PONA column. Once the GPC result shows that the 16 compounds have been degraded, a transplanting of the resultant culture (at 20%, v/v) is initiated in the MM medium by the same method previously described. This second series of cultures constitutes the Mix2. The cultures are incubated, and the residual substrates are measured as described in the preceding stage. After consumption of the substrates, a third transplanting is initiated (Mix3). Given the quantities of Mix2 available at the time of inoculation to obtain the Mix3 culture, it was not possible to measure the biomass added to the Mix3 flask. After 196 days of incubation, the results obtained show the degradation capacities described in Table 3.

TABLE 3
Degradation Capacities of the 16 Compounds of the Mix3 Culture.

| Chemical Compound | Percentage of Degradation |
|---|---|
| Octane | 100 |
| Hexadecane | 95.3 ± 0.1 |
| Isooctane | 40.4 ± 1.9 |
| Benzene | 100 |
| Toluene | 100 |
| Ethylbenzene | 98.1 ± 1.3 |
| o-Xylene | 95.6 ± 0.6 |
| m-Xylene | 97.4 ± 1.9 |
| p-Xylene | 97.8 ± 1.5 |
| Naphthalene | 97.0 ± 0.6 |
| MTBE | 34.7 ± 2.8 |
| ETBE | 100 |
| TBA | 0 |
| Cyclohexane | 94.1 ± 0.5 |
| Cyclohexanol | 100 |
| 2-EHN | 90.4 ± 1.5 |

In order to identify the microorganisms responsible for degradation, a sample of this culture is smeared in diluted form on dishes of rich TS agar (Tripticase/soy) medium. The dishes are incubated at 30° C. After growth, the individualized colonies are collected and isolated on an identical solid medium.

It has thus been possible to isolate several different bacteria including Rhodococcus wratislaviensis and Rhodococcus aetherivorans, which were deposited at the Institut Pasteur under the references CNCM I-4088 and CNCM I-4089 respectively.

### Example 2

### Degradation and Mineralization Capacities of the 16 Compounds Individually Tested by Rhodococcus wratislaviensis CNCM I-4088 and Rhodococcus aetherivorans CNCM I-4089

It is desired to determine the capacities of each of these two strains Rhodococcus wratislaviensis CNCM I-4088 and Rhodococcus aetherivorans CNCM I-4089 individually in regard to the 16 compounds.

Precultures of each of the strains Rhodococcus wratislaviensis CNCM I-4088 and Rhodococcus aetherivorans CNCM I-4089 are carried out on the rich liquid TS medium. The cultures are centrifuged and then washed twice in the MM medium described in Example 1.

1) Mineralization Tests:

They are carried out in 160 ml penicillin flasks into which 20 ml of medium is introduced. The flasks are inoculated with either Rhodococcus wratislaviensis CNCM I-4088 or with Rhodococcus aetherivorans CNCM I-4089. The quantity of biomass introduced into each of the flasks is the same for the 2 strains used and corresponds to a final optical density value at 600 nm (=OD₆₀₀) that is equal to 0.5. Then, the substrates (1 substrate/flask) are added at a rate of 5 μl/flask. In parallel, series of control flasks are prepared into which strains are introduced but without substrate. These flasks will be used to measure the endogenous respiration of each of the strains in the absence of substrate. The flasks are plugged with butyl stoppers and incubated at 30° C. for 8 weeks while being stirred. Then, 1 ml of HNO₃ (60%)/flask is added by syringe through the stopper so as to strip the CO₂ from the aqueous phase to the gaseous phase. The total CO₂ produced in each flask can then be measured by taking a sample from the headspace with a gas-tight syringe. This measurement is carried out on a GPC equipped with a katharometer. The calculation of CO₂ produced on a given substrate by each strain is done after having subtracted the value of the endogenous respiration. The calculation of the CO₂ value is carried out in relation to a gaseous standard containing CO₂ at a set concentration. The calculation of mineralization is carried out by relating the carbon found in the CO₂ to the carbon brought by the substrate. The results are presented in Table 4.

In certain cases, substrate was added after dissolution of the substrate in a third solvent (2,2,4,4,6,8,8-heptamethyl-nonane or HMN), thereby making it possible both to enhance the solubilization of the compound in the growth medium and to reduce its toxicity for the microorganisms. In this case, the quantity of substrate (5 μl) is introduced into 0.5 ml of HMN. The cases where this procedure was followed are directly indicated in Table 4.

2) Degradation Tests:

They are carried out under conditions similar to what is described in the mineralization test. In this case, the test controls consist of a series of flasks under the same conditions and containing each substrate, but not inoculated. At the conclusion of the test, the residual substrates are measured:

Either directly from a sample of the aqueous phase in the case of very soluble substrates (in the case of MTBE, ETBE, TBA and cyclohexanol). This metering is done by GPC/FID equipped with a CP PorabondQ (Varian) column.

Or after extraction with pentane containing 1,1,2-TCA as the internal standard. After extraction of the residual substrates, the pentane is injected into the GPC/FID on a PONA column.

The calculations are made in relation to the residual substrate measured in the uninoculated control flasks. The results are presented in Table 4.

TABLE 4
Degradation and Mineralization Capacity of Rhodococcus wratislaviensis CNCM I-4088
and Rhodococcus aetherivorans CNCM I-4089 on the 16 Separately-Tested Substrates.

| | R. wratislaviensis CNCM I-4088 | | R. aetherivorans CNCM I-4089 | |
|---|---|---|---|---|
| Tested | % of | % of | % of | % of |
| Compound | Degradation | Mineralization | Degradation | Mineralization |
| Benzene | 64.9 ± 0.8^{a} | 25.4 ± 6.3^{a} | 4.1 ± 16.0^{a} | ≦0^{a} |
| Ethylbenzene | 95.2 ± 2.7^{a} | 40.8 ± 29.9^{a} | 0^{a} | 18.8 ± 0.2^{a} |
| Toluene | 99.7 ± 0.1^{a} | 91.2 ± 0.4^{a} | 0^{a} | ≦0^{a} |
| m-Xylene | 0^{a} | 0^{a} | 0^{a} | ≦0^{a} |
| | 63.3 ± 50.4^{b} | 91.0 ± 12.7^{b} | | |
| p-Xylene | 0^{a} | 0^{a} | 0^{a} | 2.9 ± 0.2^{a} |
| | 52.1 ± 26.3^{b} | 4.8 ± 6.8^{b} | | |
| o-Xylene | 13.5 ± 17.2^{a} | 0^{a} | 0^{a} | 1.2 ± 3.2^{a} |
| | 48.1 ± 1.8^{b} | 29.3 ± 7.3^{b} | | |
| Cyclohexane | 0^{a} | 0^{a} | 0^{a} | ≦0^{a} |
| | 100 | 75.6 ± 11.6^{b} | | |
| Octane | 97.5 ± 0.1^{a} | 63.7 ± 10.7^{a} | 21.4 ± 23.6^{a} | 5.7 ± 17.6^{a} |
| Hexadecane | 32.1 ± 2.2^{a} | 78.1 ± 18.2^{a} | 96.3 ± 1.8^{a} | 65.0 ± 11.0^{a} |
| Isooctane | 16.8 ± 8.9^{a} | 0^{a} | 17.3 ± 4.6^{a} | 1.0 ± 4.8^{a} |
| Cyclohexanol | 100^{a} | 69.7 ± 4.0^{a} | 100^{a} | 77.1 ± 18.6^{a} |
| MTBE | 3.2 ± 0.1^{a} | 0^{a} | 22.3 ± .2^{a} | ≦0^{a} |
| | (production of TBA) | | (production of TBA) | |
| ETBE | 0^{a} | 0^{a} | 100^{a} | 25.0 ± 3.7^{a} |
| | | | (production of TBA) | |
| TBA | 0^{a} | 0^{a} | 0^{a} | ≦0^{a} |
| 2-EHN | 0^{b} | 0^{b} | 26.2 ± 0.6^{a} | 7.5 ± 15.7^{a} |
| Naphthalene | 100 | 52.5 ± 11.0^{a} | 26.3 | ≦0^{a} |

^{a}The substrates were directly introduced into the medium.
^{b}The substrates were introduced after dissolution in HMN.

In certain cases, degradation is not total even though it was so when the strain was tested on the compounds provided in the mixture (the case, for example, of Rhodococcus wratislaviensis CNCM I-4088 tested on benzene). Therefore, the fact that the concentrations are different in these two experiments must be taken into account: when the benzene is tested individually, it is added at a final concentration of 220 mg.L⁻¹, whereas, in the compound mixture, it is provided at 7.2 mg.L⁻¹ (see Table 2).

A second comment is that the addition of m-xylene alone, p-xylene alone, o-xylene alone or of cyclohexane alone at higher concentrations (215, 215, 220 or 195 mg.L⁻¹, respectively) does not allow biodegradation by Rhodococcus wratislaviensis CNCM I-4088. In contrast, these compounds are degraded when the same quantity of each of them (5 μl) is introduced into a third solvent such as HMN. This is well illustrated in Table 4.

In certain cases, there is total biodegradation of compounds and a small percentage of mineralization: this is the case, for example, of ETBE by Rhodococcus aetherivorans CNCM I-4089: this is explained by the fact that only the C2 fragment released through cleavage of the ETBE ether bond is used as a substrate by the bacterium, and the mineralization yield is calculated in relation to the total quantity of ETBE introduced into the flask (5 μl).

### Example 3

### Degradation Capacities of the 16 Compounds in a Mixture by Rhodococcus wratislaviensis CNCM I-4088, by Rhodococcus aetherivorans CNCM I-4089, and by a Co-Culture of the 2 Strains

Precultures of Rhodococcus wratislaviensis CNCM I-4088 and Rhodococcus aetherivorans CNCM I-4089 are made in the TS medium. After centrifuging and washing as described in Example 2, the strains Rhodococcus wratislaviensis CNCM I-4088 and Rhodococcus aetherivorans CNCM 1-4089 are tested for their capacities for degrading the mixture of 16 compounds under the conditions described in Example 1, with the strains being tested separately, and then in co-culture. The biomass introduced into the experiments regarding the individually tested strains corresponds to an OD₆₀₀ value of 0.5. When the mixture of two strains is involved, a suspension containing each strain is made up at the same cellular concentration, and the flasks are inoculated with this mixture in such a way as to obtain an OD₆₀₀ of 0.5 as well. After incubation at 30° C., the residual substrates are metered after 4 weeks of incubation as previously described. The results are presented in Table 5.

TABLE 5
Degradation of the Mixture of 16 Substrates by Rhodococcus wratislaviensis
CNCM I-4088, by Rhodococcus aetherivorans CNCM I-4089,
or by a Co-Culture of these Two Strains

| Compounds | Degradation | Degradation | Degradation Capacity of the |
|---|---|---|---|
| Present in the | Capacity of | Capacity of | Mixture Rhodococcus |
| Substrate | Rhodococcus | Rhodococcus | wratislaviensis and |
| Mixture | wratislaviensis | aetherivorans | Rhodococcus aetherivorans |
| Benzene | 100 | 5.7 ± 0.02 | 100 |
| Ethylbenzene | 100 | 24.0 ± 2.2 | 97.5 ± 0.3 |
| Toluene | 100 | 2.0 ± 1.0 | 100 |
| m-Xylene | 100 | 2.4 ± 4.3 | 95.9 ± 0.4 |
| p-Xylene | 100 | 0 | 96.0 ± 0.3 |
| o-Xylene | 100 | 1.7 ± 2.4 | 96.0 ± 0.3 |
| Cyclohexane | 100 | 5.1 ± 1.0 | 100 |
| Octane | 94.3 ± 1.4 | 9.0 ± 1.2 | 91.6 ± 0.3 |
| Hexadecane | 100 | 96.5 ± 4.4 | 98.1 ± 2.7 |
| Isooctane | 26.3 ± 10.7 | 2.6 ± 1.2 | 29.8 ± 1.6 |
| Cyclohexanol | 100 | 100 | 100 |
| MTBE | 78.2 ± 1.3 | 32.4 ± 0.1 | 100 |
| ETBE | 50.8 ± 1.4 | 100 | 100 |
| TBA | No degradation | No degradation | No degradation |
| | (Production of TBA | (Production of TBA | (Production of TBA |
| | by degradation of | by degradation of | by degradation of |
| | MTBE and ETBE) | MTBE and ETBE) | MTBE and ETBE) |
| 2-EHN | 67.9 ± 13.3 | 37.8 ± 4.7 | 72.9 ± 1.7 |
| Naphthalene | 100 | 5.5 ± 7.3 | 97.8 ± 0.1 |

### Example 4

### Degradation Capacities of the 16 Compounds in a Mixture by a Co-Culture Made up of Rhodococcus wratislaviensis CNCM I-4088, Rhodococcus aetherivorans CNCM I-4089, and Aquincola tertiaricarbonis IFP2003

Aquincola tertiaricarbonis CNCM I-2052 was previously isolated for its capacities to degrade TBA. It was thus advantageous to test it in association with the two strains Rhodococcus wratislaviensis CNCM I-4088 and Rhodococcus aetherivorans CNCM I-4089 in order to degrade the TBA that is not consumed by these two strains, but, on the contrary, produced during the degradation of MTBE and ETBE.

Precultures of Rhodococcus wratislaviensis CNCM I-4088, Rhodococcus aetherivorans CNCM I-4089, and Aquincola tertiaricarbonis CNCM I-2052 are produced in the TS medium. After centrifuging and washing as described in Example 2, a co-culture containing the 3 strains is prepared and then tested for its capacities to degrade the mixture of 16 compounds under the conditions described in Example 1. With the biomass having been introduced in the experiments regarding the mixture of 3 strains, a suspension containing each strain at the same cellular concentration is made up, and the flasks are inoculated with this mixture so as to obtain an OD₆₀₀ of 0.5 as well. After 4 weeks of incubation at 30° C., the residual substrates are metered as described previously, and the results are presented in Table 6.

TABLE 6
Degradation of the Mixture of 16 Substrates by a Co-
Culture Made up of Rhodococcus wratislaviensis CNCM
I-4088, Rhodococcus aetherivorans CNCM I-4089, and
Aquincola tertiaricarbonis CNCM I-2052.

| | Degradation Capacity of the Co-Culture Made up of |
|---|---|
| Compounds | Rhodococcus wratislaviensis I-4088, Rhodococcus |
| Present in the | aetherivorans I-4089 and Aquincola |
| Substrate Mixture | tertairicarbonaris IFP2003 |
| Benzene | 100 |
| Ethylbenzene | 98.1 ± 2.6 |
| Toluene | 100 |
| m-Xylene | 96.7 ± 4.6 |
| p-Xylene | 97.2 ± 3.9 |
| o-Xylene | 97.2 ± 4.0 |
| Cyclohexane | 100 |
| Octane | 94.2 ± 2.0 |
| Hexadecane | 97.9 ± 3.0 |
| Isooctane | 32.0 ± 2.8 |
| Cyclohexanol | 100 |
| MTBE | 100 |
| ETBE | 100 |
| TBA | 100 |
| 2-EHN | 80.0 ± 12.7 |
| Naphthalene | 98.0 ± 2.9 |

1. Process for treatment of aqueous effluents comprising a complex mixture of substances containing native hydrocarbons of gasolines and additives that are present in gasolines or diesel fuel, in which at least one bacterium selected from among the bacteria Rhodococcus wratislaviensis CNCM I-4088 and Rhodococcus aetherivorans CNCM I-4089 is grown under aerobic conditions in the presence of a growth substrate containing said mixture as a carbon source, and said mixture is at least partially degraded by the bacterium down to the final degradation products—carbon dioxide, water, and biomass.

2. Process according to claim 1, in which the mixture includes compounds selected from among alkanes, monoaromatic hydrocarbons, polycyclic aromatic hydrocarbons, ethers or nitrates.

3. Process according to claim 2, in which the mixture includes octane, hexadecane, benzene, ethylbenzene, toluene, m-xylene, p-xylene, o-xylene, cyclohexanol, tert-butanol (hereafter referred to by the term TBA), cyclohexane, isooctane, MTBE, ETBE, 2-ethyl hexyl nitrate (hereafter referred to by the term 2-EHN), and naphthalene.

4. Process according to claim 1, in which the two bacteria Rhodococcus wratislaviensis CNCM I-4088 and Rhodococcus aetherivorans CNCM I-4089 are grown under aerobic conditions in the presence of a growth substrate containing said mixture as a carbon source, and said mixture is at least partially degraded by the bacteria down to the final degradation products—carbon dioxide, water and biomass.

5. Process according to claim 1, in which a consortium containing the three bacteria Rhodococcus wratislaviensis CNCM I-4088, Rhodococcus aetherivorans CNCM I-4089, and Aquincola tertiaricarbonis CNCM I-2052 is grown under aerobic conditions in the presence of a growth substrate containing said mixture as a carbon source, and said mixture is at least partially degraded by the bacteria down to the final degradation products—carbon dioxide, water, and biomass.

6. Process according to claim 1, in which the bacterium or the bacterial consortium is developed on a mineral or organic substrate in a biofilter or biobarrier system of adequate volume, effluents to be treated in the presence of air or oxygen are introduced into the biofilter or biobarrier, and the effluent is drawn off with a reduced concentration of chemical substances.

7. Process according to claim 1, in which the bacterium or the bacterial consortium is added as inoculum to waste water purification plant sludge.

8. New bacterium Rhodococcus wratislaviensis deposited at the Institut Pasteur under the number CNCM I-4088.

9. New bacterium Rhodococcus aetherivorans deposited at the Institut Pasteur under the number CNCM I-4089.
